(19) United States
(12) Patent Application Publication
(10) Pub. No.: US 2023/0111815 A1
(43) Pub. Date: Apr. 13, 2023

(54) DEVICES WITH IMPROVED ANTIBACTERIAL SURFACE

(71) Applicant: PULSE IP, LLC, QUAKERTOWN, PA (US)

(72) Inventor: Shahram Amini, Berwyn, PA (US)

(21) Appl. No.: 17/963,653

(22) Filed: Oct. 11, 2022

Related U.S. Application Data
(60) Provisional application No. 63/254,752, filed on Oct. 12, 2021.

(51) Int. Cl. A61L 27/30; A61L 27/54; A61L 31/08; A61L 31/16

(52) U.S. Cl. CPC A61L 27/306; A61L 27/54; A61L 31/088; A61L 31/16; A61L 2300/102; A61L 2300/104; A61L 2300/404; A61L 2420/02

(57) ABSTRACT

A medical device includes a substrate structure with a surface. The surface is laser treated to define at least one protrusion and/or at least one void extending relative to the surface. A coating having antibacterial, antimicrobial and/or drug eluding properties is applied to the substrate structure such that the coating engages within or along a surface portion of one or more of the protrusions and/or voids.

This application claims the benefit of U.S. Prov. Appln. No. 63/254,752, filed on Oct. 12, 2021, the contents of which are incorporated herein by reference.

### FIELD OF THE DISCLOSURE

The present disclosure relates to electrodes and biomedical, implantable or diagnostic medical devices. The devices have an improved surface topography which supports a coating which imparts antibacterial, antimicrobial, or drug eluting properties.

### BACKGROUND

Ageing population and a multitude of neurological and cardiovascular illnesses that cannot be mitigated by medication alone have resulted in a significant growth in the number of patients that require implantable neurostimulation devices. These range from sensors, gastric and cardiac pacemakers, cardioverter defibrillators, to deep brain, nerve, and bone stimulators. Resorbable electronics may offer excellent short-term performance without the need for surgical removal. However, most electronic materials have poor bio- and cytocompatibility, resulting in immune reactions and infections.

Infection is a serious complication of devices implanted in the human body. Deep infections, which are difficult to treat may often require removal of the infected implant to eradicate infection and this remains a serious complication of many medical procedures. Treatment of deep infections is challenging because it is difficult to supply antibiotics to the infection site, and such treatment can vary from 3 to 14 months in duration and can include secondary surgery.

Others have previously recognized the antimicrobial properties of metals and metal oxides, especially in a micro- or nanoparticulate form, and there is extensive literature reporting that effect. A common shortcoming of known metal containing coatings is that efforts to improve adherence of such coatings (e.g., to provide durability and scratch-resistance) can adversely affect their antimicrobial properties. Conversely, efforts to improve the antimicrobial efficacy of such coatings can adversely affect their ability to stably adhere to implant surfaces.

### SUMMARY

Hierarchical surface restructuring (HSR™) technology is capable of fabricating hierarchically structured surfaces (HSS) on microelectrodes for ultrahigh surface area and enhanced electrochemically-active-surface-area. However, the electrode materials (e.g., Pt10Ir) or common surface coatings (e.g. TiN or IrO₂) that are deposited onto the electrode materials may not be antimicrobial. On the other hand, highly effective broad-spectrum antimicrobial materials, e.g., CuₓO, are insulators with poor electrochemical properties and cannot be used as electrodes. Coating electrodes or microelectrode arrays that are hierarchically restructured on the surface with atomically thin and ultra-conformal antimicrobial material may impart antimicrobial property to the electrode or microelectrode array.

Here, the atomically thin thickness is essential for minimal effect on HSS' nanoscale morphology and functionalities (e.g., ultrahigh surface area, charge storage capacity, impedance and specific capacitance). The ultra-conformality is essential for the complete antimicrobial coverage for the complex nanostructured HSS. Here, the two essential features—ultra-conformality and atomically thin thickness—are extremely challenging for conventional coating techniques (e.g., sputtering, PVD, and CVD) due to their (1) line-of-sight effect and (2) difficulty of atomic-thickness control on sub-100 nm nanocoatings. On the contrary, atomic layer deposition (ALD) coating technique is ideal (the only one) to achieve the two essential features and ultrahigh repeatability due to its intrinsically self-limiting coating mechanism. Moreover, ALD technique has been used in modern applications with automated high parallel processing throughput.

In at least one embodiment, the present disclosure provides a medical device including a substrate structure with a surface. The surface is laser treated to define at least one protrusion and/or at least one void extending relative to the surface. A coating having antibacterial, antimicrobial and/or drug eluding properties is applied to the substrate structure such that the coating engages within or along a surface portion of one or more of the protrusions and/or voids.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIGS. 1A-1C show SEMs illustrating an exemplary substrate structure obtained according to an embodiment of the invention.

FIG. 2 illustrates an exemplary surface topography with voids therein.

FIG. 3 is a schematic drawing illustrating an exemplary substrate structure having multiple projections and voids.

FIG. 4 is a schematic drawing illustrating an embodiment of the disclosure.

FIG. 5 is a schematic drawing illustrating another embodiment of the disclosure.

FIG. 6 is a schematic drawing illustrating yet another embodiment of the disclosure.

FIG. 7 is a schematic drawing illustrating yet another embodiment of the disclosure.

FIG. 8 is a schematic drawing illustrating yet another embodiment of the disclosure.

FIGS. 9A and 9B are SEM images of PEALD CuₓO coated Pt10Ir (FIG. 9A) and Pt10Ir HSS (FIG. 9B) samples.

FIGS. 10A-10C show EDS spectra and compositional analysis of PEALD CuO thin films on Silicon substrate (FIG. 10A), Pt10Ir substrate (FIG. 10B) and Pt10Ir HSR substrate (FIG. 10C).

FIG. 11 shows XPS results of the three PEALD-coated samples: silicon, Pt10Ir, and Pt10Ir HSS substrates.

FIGS. 12A-12D show SEM images CuO of coated Si (FIG. 12A), Pt10Ir (FIG. 12B), Pt10Ir HSS (FIGS. 12C-12D) samples.

FIG. 13 shows XPS results of the three PEALD-coated samples: silicon, Pt10Ir, and Pt10Ir HSS substrates.

FIGS. 14A-14D show AFM scanning of O3-ALD coated masked Si sample at the boundary with FIG. 14A an AFM scanning showing tip and the masking boundary. FIG. 14B a 3D view, FIG. 14C a 2D top-view, and FIG. 14D a 2D profile at the boundary.

FIGS. 15A-15D are optical images of the mask-coated HSS sample, clearly showing the boundary of coated and uncoated/masked regions with FIG. 15C showing the coated region.

FIGS. 16A-16E are SEM images of CuO coated HSS sample. FIGS. 16A-16C show the boundary around the coated and uncoated region, FIG. 16D is of masked region and FIG. 16E represents surface structure in the coated region.

FIGS. 17A-17F show EDS mapping around the interface of masked and unmasked region of HSS samples.

FIG. 18 shows ICP-MS analysis of uncoated Si substrates vs. media (water). The Y-axis represents the cumulative release of Cu ions as detected by ICP-MS.

FIG. 19 shows ICP-MS analysis of uncoated Si substrates vs. media (sterile LB growth media). The Y-axis represents the cumulative release of Cu ions as detected by ICP-MS.

FIG. 20 shows ICP-MS analysis of CuO coated “flat” and HSS substrates vs uncoated substrates in distilled water. The Y-axis represents the cumulative release of Cu ions as detected by ICP-MS.

FIG. 21 shows ICP-MS analysis of CuO coated “flat” and HSS Pt substrates vs uncoated substrates in sterile LB media. The Y-axis represents the cumulative release of Cu ions as detected by ICP-MS.

FIG. 22 shows agar plates after swabbing inoculated CuO coated Si substrates with (A) showing S. aureus and (B) showing E. coli. The images are representative of duplicates.

FIG. 23 shows OD600 measurements of liquid cultures inoculated from Si substrates +/−CuO coatings. These cultures were incubated for ˜18 h after the initial inoculation. PBS in included as a negative control.

FIG. 24 shows agar plates after swabbing inoculated “flat” Pt uncoated substrates with (A) showing S. aureus and (B) showing E. coli. The images are representative of duplicates.

FIG. 25 shows agar plates after swabbing inoculated Pt HSS uncoated substrates with (A) showing S. aureus and (B) showing E. coli. The images are representative of duplicates.

FIG. 26 shows agar plates after swabbing inoculated “flat” Pt, CuO coated substrates with (A) showing S. aureus and (B) showing E. coli. The images are representative of duplicates.

FIG. 27 shows agar plates after swabbing inoculated Pt HSS, CuO coated substrates with (A) showing S. aureus and (B) showing E. coli. The images are representative of duplicates.

FIG. 28 shows OD600 measurements of liquid cultures inoculated from uncoated Pt substrates, either “flat” or HSS surfaces. These cultures were incubated for ˜18 h after the initial inoculation. PBS is included as a negative control.

FIG. 29 shows OD600 measurements of liquid cultures inoculated from CuO coated Pt substrates, either “flat” or HSS surfaces. These cultures were incubated for ˜18 h after the initial inoculation. PBS is included as a negative control.

### DETAILED DESCRIPTION

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

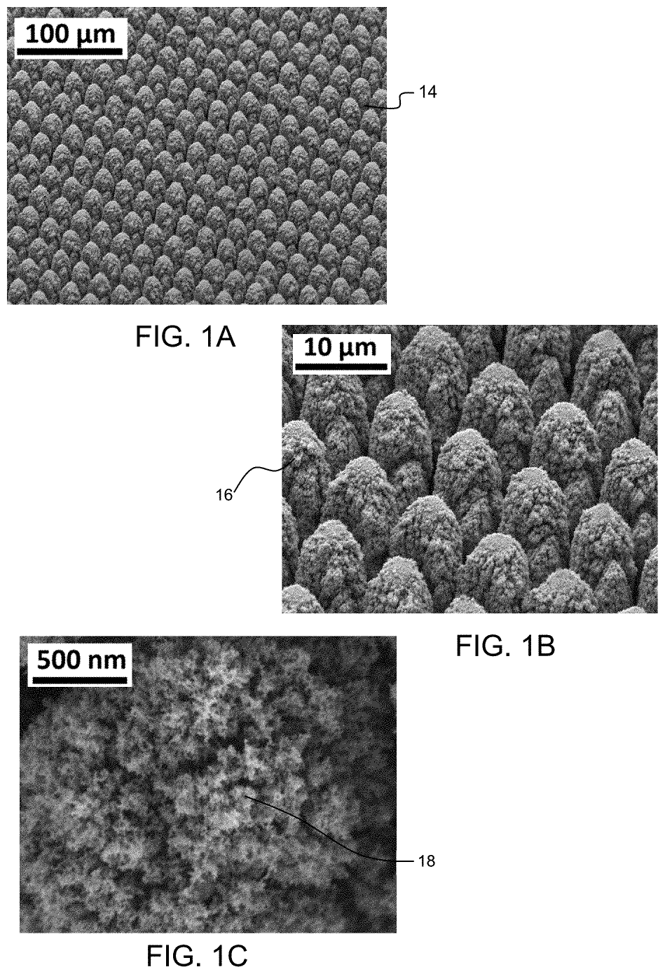

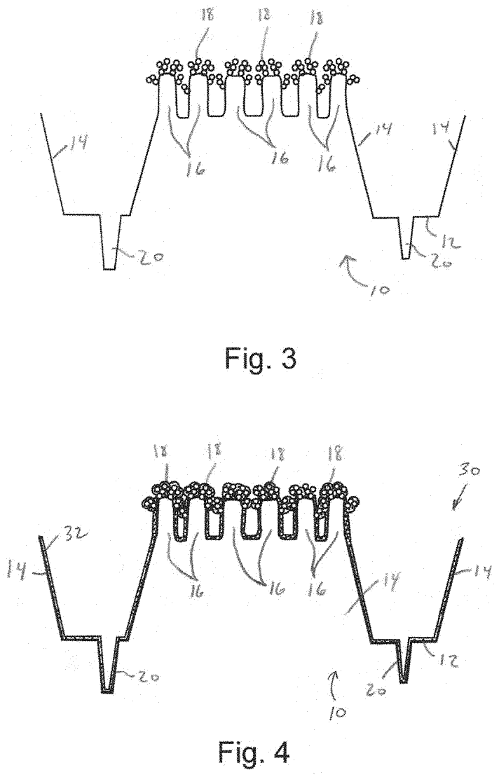

Referring to FIGS. 1A-3, an illustrative medical device substrate structure 10 in accordance with an embodiment of the disclosure will be described. Potential medical devices include, but are not limited to, electrodes, microelectrode arrays, stents, orthopedic and dental implants, etc. The substrate structure 10 and the coating 32 of the medical device 30 may be selected to provide a combination of desired structural and antibacterial properties. The illustrated substrate structure 10 has a surface 12 topography defined by a plurality of macro protrusions 14, micro protrusions 16 and nano protrusions 18. The surface 12 may also include a plurality of voids 20. While described as an illustrative structure, the substrate structure 10 of the medical device 30 may have more or fewer protrusions and may or may not include voids.

In the illustrated embodiment, the outer peripheral surface has a topography defined by a plurality of discrete macro protrusions 14 distributed about and extending outwardly from the outer peripheral surface 12 (see FIG. 1A). In one embodiment, the macro protrusions 14 are substantially uniformly distributed across the outer peripheral surface of the solid, monolithic substrate. In one embodiment, the macro protrusions have a width in the range of from about 0.15 μm to about 50 μm. In another embodiment, the macro protrusions have a width in the range of from about 0.2 μm to about 30 μm. In yet another embodiment, the macro protrusions have a width in the range of from about 1 μm to about 20 μm.

A plurality of discrete micro protrusions 16 are distributed on and extend outwardly from the macro protrusions 14 (see FIG. 1B). In one embodiment, the micro protrusions 16 have a width ranging from about 0.15 μm to about 5 μm. In another embodiment, the micro protrusions 16 have a width in the range of from about 0.2 μm to about 2 μm. In yet another embodiment, the micro protrusions 16 have a width in the range of from about 0.4 μm to about 1.5 μm. In one embodiment the micro protrusions 16 are distributed across the macro protrusions 14 in the form of periodic waves of the heights of the micro protrusions. It is believed that the periodic waves are caused and controlled by the wavelength of the laser irradiation.

A plurality of discrete nano protrusions 18 are distributed on and extending outwardly from the micro protrusions 16 (see FIG. 1C). In one embodiment, the nano protrusions 18 have a width ranging from about 0.01 μm to about 1 μm. In another embodiment, the nano protrusions 18 have a width in the range of from about 0.02 μm to about 1 μm. In yet another embodiment the nano protrusions 18 have a width in the range of from about 0.075 μm to about 0.8 μm. In one embodiment, the nano protrusions 18 are distributed across the micro protrusions 16 in the form of tubes and/or globules. It is believed that the nano protrusions 18 are caused and controlled by the number of pulses and the pulse duration. Without being held to a particular theory, it is believed that the macro, micro and nano protrusions are formed by the laser drilling voids in the substrate surface, and then the materials from the voids are re-deposited onto the substrate surface as these protrusions. It is therefore important that the laser irradiation is done without purging the substrate with a gas and without any substantial gas pressure since such would tend to blow the void material away rather than re-depositing it onto the substrate. It is believed that the atmosphere in which laser irradiation is conducted is generally not important as long as the removed void material is not blown away, and is allowed to re-deposit onto the substrate. However, in some instances the atmosphere may be important, for example, for materials like Ti, Nitrogen is needed to react with Ti to form electrochemically active high surface area TiN. The drilling effect is most intense at the center of the laser spot, and therefore the traversing of the laser spot across the substrate surface causes an overlapping of spots, and therefore a Gaussian distribution of applied laser radiation.

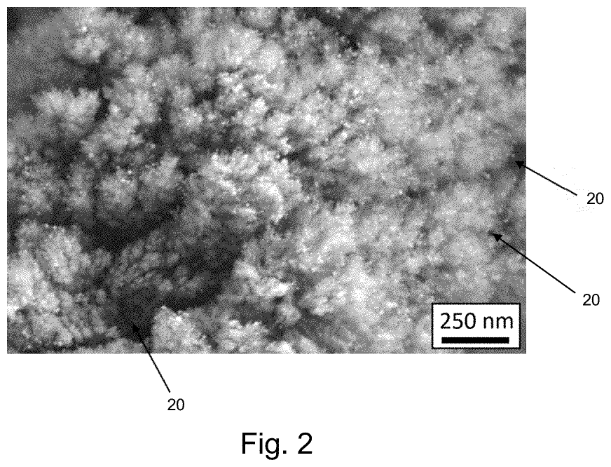

In another embodiment of the invention, in addition to these discrete macro, micro, and nano protrusions which extend outwardly from the substrate surface, the surface structure 12 may have a laser induced array of voids 20 (see FIG. 2) whose length and depth depend on the laser parameters employed. Thus, in this embodiment, the outer peripheral surface additionally has a topography with a plurality of voids 20 distributed about the outer peripheral surface which extending a depth through the substrate. The voids have a depth through the substrate of from about 50 nm to about 500 nm, preferably from about 100 nm to about 250 nm. The voids have a width of from about 50 nm to about 500 nm, preferably of from about 100 nm to about 250 nm. The voids are spaced from adjacent voids a distance of from about 50 nm to about 250 nm.

A substrate surface 10 according to the disclosure, is produced by exposing an outer peripheral surface of a solid, monolithic substrate of a biocompatible metal to pulses of laser irradiation. In one embodiment the laser has a spot diameter ranging from about 1 μm to about 1000 μm. In another embodiment, the laser has a spot diameter ranging from about 2 μm to about 250 μm, and in yet another embodiment, the laser has a spot diameter ranging from about 5 μm to about 200 μm. In one embodiment the number of pulses of laser irradiation per spot, ranges from about 10 to about 1500 pulses. In another embodiment, the number of pulses of laser irradiation per spot ranges from about 20 to about 1000, and in yet another embodiment, the number of pulses of laser irradiation per spot ranges from about 100 to about 500. In one embodiment the laser has a pulse wavelength which ranges from about 200 nm to about 1500 nm. In another embodiment, the pulse wavelength ranges from about 400 to about 1,000, and in yet another embodiment, the pulse wavelength ranges from about 400 to about 800. In one embodiment the laser pulse width ranges from about 1 femtosecond to about 5 picoseconds. In another embodiment the laser pulse width ranges from about 1 femtosecond to about 3 picoseconds. In one embodiment the laser irradiance ranges from about 200 Watts/cm² to about 5000 Watts/cm². The exposing may be conducted by traversing the spot of laser radiation across the outer peripheral surface of the solid, monolithic substrate at a rate of from about 50 mm/min to about 1000 mm/min, however, the rate is not critical to the invention and only affects the cost-effective execution of the inventive method.

Examples of suitable lasers non-exclusively include a Coherent Libra-F Ti:Sapphire amplifier laser system, a Rofin Startfemto, and a Coherent AVIA laser. According to the disclosure, the resulting electrode has a polarization of about 1,000 mV or less, preferably about 500 mV or less, and more preferably about 200 mV or less.

Referring to FIG. 4, the medical device 30 includes a coating 32 applied to the entire surface 12 of the substrate structure 10. The coating 32 may have various properties, for example, antibacterial and/or antimicrobial properties. The coating 32 may also have drug eluding properties, for example, anti-cancer drugs, and/or antibacterial or antimicrobial drugs.

Antibacterial coatings or thin films that contain bactericidal elements such as zinc, copper and/or silver, are known to have bactericidal properties when in ion form. Most of these elements form an oxide when exposed to oxygen under specific processing and/or operating conditions and their oxides can also provide antibacterial properties. Examples include zinc oxide, silver oxide, and copper oxide.

While basic coatings are known, the laser restructuring or texturing of the substrate structure 10 configures the surface 12 such that the coating has the greatest efficacy. For example, in the embodiment illustrated in FIG. 4, the protrusions 14, 16, 18 and voids 20 define an increased surface area, thereby facilitating a maximum amount of coating.

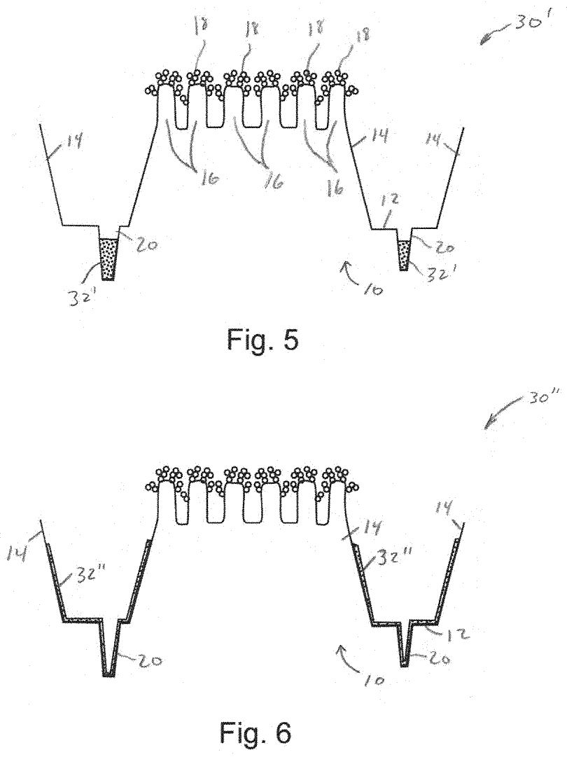

Turning to the medical device 30′ illustrated in FIG. 5, the coating 32′ is only applied within the voids 20. With such a configuration, the coating 32′ has minimal exposure and may be utilized in applications where a slow, extended release is desired. In the embodiment illustrated in FIG. 6, the coating 32″ of medical device 30″ is applied along the surface 12 between the macro protrusions 14 and within the voids 20. Compared to the previous embodiment, the coating 32″ is exposed along more surface area and therefore may be released more quickly, however, is still below the outer extensions of the protrusions 16, 18 and is therefore protected.

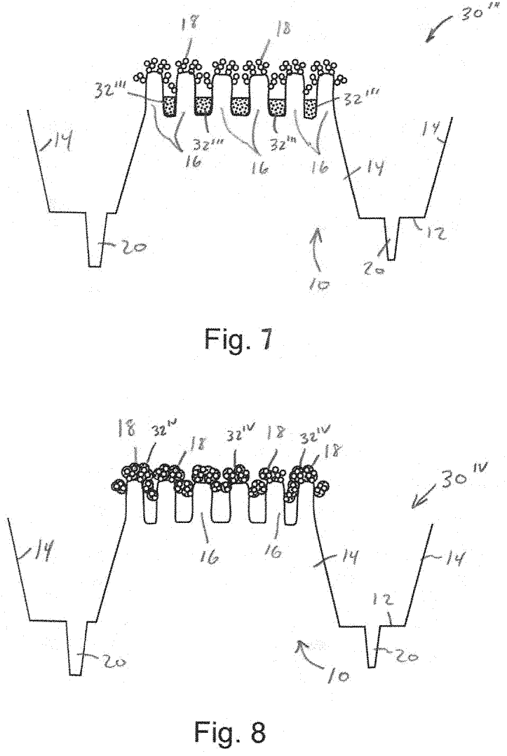

With the medical device 32′″ illustrated in FIG. 7, the coating 32′″ is applied along the surface 12 in between the micro protrusions 16. With this configuration, the coating 32′″ is proximate the outer surface for contact and more rapid release, however, is still protected by the nano protrusions 18 extending further outward. Conversely, in the medical device 32^{iv} illustrated in FIG. 8, the coating 32^{iv} is applied to the nano protrusions 18 only. This configuration provides the greatest exposure for the coating 32^{iv} and a corresponding rapid release.

Sample structures incorporating various features described above were manufactured and tested as explained in more detail hereinafter. More specifically, ultra-conformal atomically thin CuₓO antimicrobial coatings were fabricated on silicon, flat Pt10Ir, and Pt10Ir HSS via ALD. As one example, a Veeco Fiji Plasma Enhanced ALD (PEALD), which is capable of depositing thermal or plasma enhanced ALD, was used to deposit CuₓO ALD coatings.

The samples were exposed to repeating cycles of Cu-containing precursor as well as oxidation reactants (e.g., O₃, O plasma, or H₂O). For each cycle, the Cu-containing precursor and gas reactant(s) react with the substrates at a time in a sequential, self-limiting manner. Thus, an ultra-conformal CuₓO pre cycle would grow on the samples. The quality and properties of the CuₓO ALD coating have been characterized by various characterization techniques: thickness and optical properties by ellipsometer and atomic force microscope (AFM), microstructure and nanomorphology by high resolution scanning electron microscope (SEM) and AFM, microscale chemical probing by energy dispersive spectroscopy (EDS), chemical and composition by X-ray photoelectron spectroscopy (XPS), crystal structure and composition by grazing incidence X-ray diffraction (GIXRD), coating conformity and uniformity by optical microscope and SEM, and mechanical durability by nanoindenter. Based on XPS, it is confirmed that, via PEALD using O plasma and thermal ALD using O₃, the ALD coating is CuO.

As explained in more detail hereinafter, the antimicrobial properties of the coated samples have been investigated. While the uncoated Si and flat Pt10Ir have no antimicrobial properties, the uncoated Pt10Ir HSS samples interestingly demonstrate certain intrinsic antimicrobial property, possibly due to the nanoscale surface sharpness. The CuO-coated samples all demonstrated different degrees of antimicrobial property. The CuO-coated HSS samples show the highest antimicrobial property.

Coating Procedures

CuO films were grown using a Veeco Fiji PEALD system. (Bis(dimethylamino-2-propoxide) copper (II)) was used as the Cu-containing precursor, and the precursor source was maintained at 125° C. using a Veeco Low Vapor Pressure Delivery (LVPD) module. Argon gas was used as carrier gas with a constant flow rate of 30 sccm.

Two ALD deposition conditions were studied: (1) plasma enhanced ALD (PEALD) using oxygen plasma as the co-reactant, and (2) thermal ALD using Ozone (O3) as the co-reactant. The substrate temperature was maintained at 150° C. For the PEALD, each ALD cycle consisted of a 2-sec Cu precursor pulse and then a 10-sec oxygen plasma pulse, and the growth per cycle (GPC) is ˜0.05 nm. For O3-based ALD, each ALD cycle consisted of a 2-sec Cu precursor pulse and then a 0.075-sec O3 pulse, and the growth per cycle (GPC) is ˜0.02 nm.

Three types of substrates were used: atomically flat silicon, as-received Pt10Ir, and laser-processed Pt10Ir HSS. In order to analyze the ALD coating thickness, Kapton tape was used to block/mask the ALD coatings, so that only the unmasked region can be ALD-coated.

Material Characterization

A J.A.Woollam M-2000 spectroscopic ellipsometer was used to analyze the thickness and optical properties of ALD coatings. An Olympus microscope was used to characterize the sample morphology. A Hitachi S-4800 scanning electron microscope (SEM) with EDS module was used to characterize the nanoscale morphology and also EDS compositional mapping. A Versa Probe 5000 XPS was used for XPS compositional analysis; the XPS spot size was 200 μm and calibration was performed using C—C component of C is peak at 284.8 eV. A Park System AFM was used to analyze surface morphology and the film thickness for the masked-Si sample.

Results and Discussion

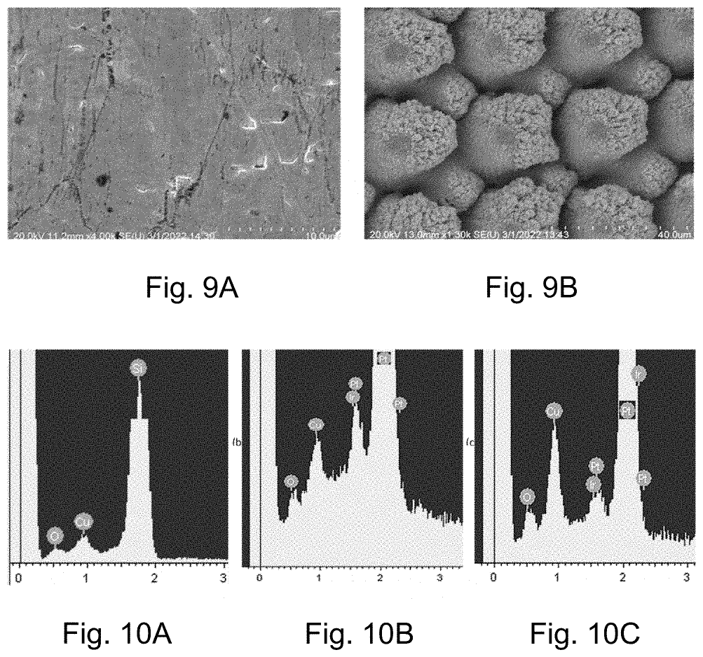

PEALD of CuO—225 cycles of PEALD CuₓO were conducted on the three types of substrates. Ellipsometry was used to test the film thickness as 12 nm, indicating a growth rate of ˜0.05 nm/cycle. FIGS. 9A and 9B show the SEM images of the coated Pt10Ir and Pt10Ir HSS samples, suggesting minimal change on the surface morphology before and after the PEALD deposition and implying nice film conformity on the sample surfaces.

EDS spectra as in FIGS. 10A-10C indicates Cu and O as expected for CuₓO deposition on all three kinds of substrates. As shown in Table 1 below, quantitative EDS analysis indicates that the surface compositions of Cu are 4.8 wt % for silicon sample, 3.9 wt % for Pt10Ir, and 13.7 wt % for Pt10Ir HSS sample.

TABLE 1
Compositional analysis of PEALD on (a) Silicon
(b) Pt10Ir and (c) Pt10Ir HSS Surface

| | | |
|---|---|---|
| Element | wt. % | At. % |

(a)

| | | |
|---|---|---|
| O K | 2.32 | 4.10 |
| Cu L | 4.81 | 2.14 |
| Si K | 92.88 | 93.75 |

(b)

| | | |
|---|---|---|
| O K | 1.58 | 15.30 |
| Cu L | 3.89 | 9.46 |
| Ir M | 24.96 | 20.09 |
| Pt M | 69.57 | 55.15 |

(c)

| | | |
|---|---|---|
| O K | 3.89 | 27.55 |
| Cu L | 13.66 | 24.36 |
| Ir M | 20.62 | 12.16 |
| Pt M | 61.84 | 35.93 |

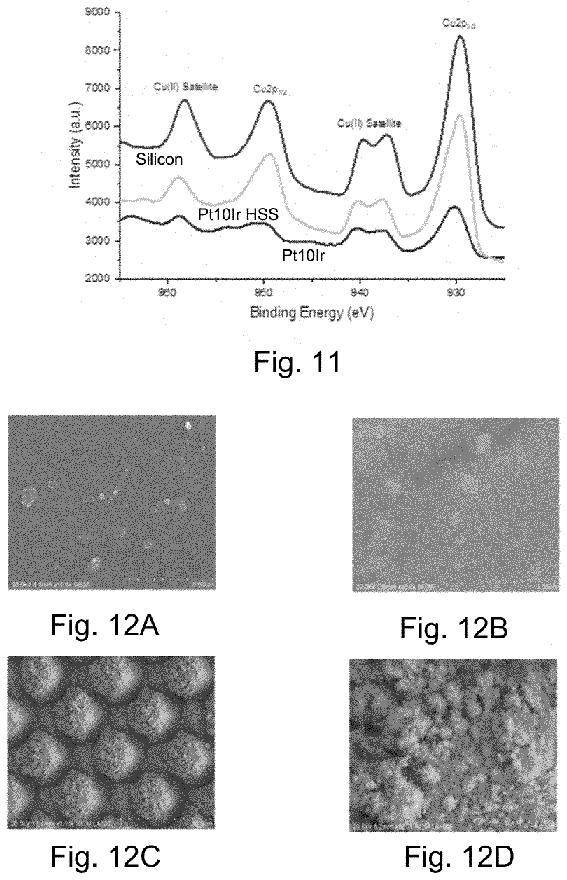

FIG. 11 shows the XPS Cu2p spectra for the PEALD thin films, clearly indicating Cu2+, i.e., the deposition if CuₓO with x=2 as CuO. For all the samples, Cu2p3/2, Cu2p1/2 and Cu2+ satellite peaks are observed at around 929.6 eV, 949.8 eV, 958.5 eV, 937.0-939.9 eV.

O3-ALD of CuO—950 cycles of O3-based ALD deposition were conducted on the three types of substrates. Ellipsometry was used to test the film thickness as 22 nm, indicating a growth rate of ˜0.05 nm/cycle. FIG. 12 shows the SEM images of the coated Si, Pt10Ir and Pt10Ir HSS samples, suggesting minimal change on the surface morphology before and after the O3-ALD.

EDS spectra (not shown) also indicates Cu and O as expected for CuₓO deposition on all three kinds of substrates. As shown in Table 2 below, quantitative EDS analysis as in Table 1 indicates that the surface compositions of Cu are 69.2 wt % for silicon sample, 2.5 wt % for Pt10Ir, and 31.5 wt % for Pt10Ir HSS sample. It should be noted that these are surface compositions of a coated surface.

TABLE 2
Compositional analysis of O3ALD on (a) Silicon
(b) Pt10Ir and (c) Pt10Ir HSS Surface

| | | |
|---|---|---|
| Element | wt. % | At. % |

(a)

| | | |
|---|---|---|
| O K | 26.92 | 57.80 |
| Cu L | 69.15 | 37.39 |
| Si K | 3.93 | 4.81 |

(b)

| | | |
|---|---|---|
| O K | 5.08 | 38.08 |
| Cu L | 2.54 | 4.80 |
| Ir M | 31.89 | 19.91 |
| Pt M | 60.49 | 37.21 |

(c)

| | | |
|---|---|---|
| O K | 11.51 | 47.69 |
| Cu L | 31.52 | 32.88 |
| Ir M | 14.36 | 4.95 |
| Pt M | 42.61 | 14.48 |

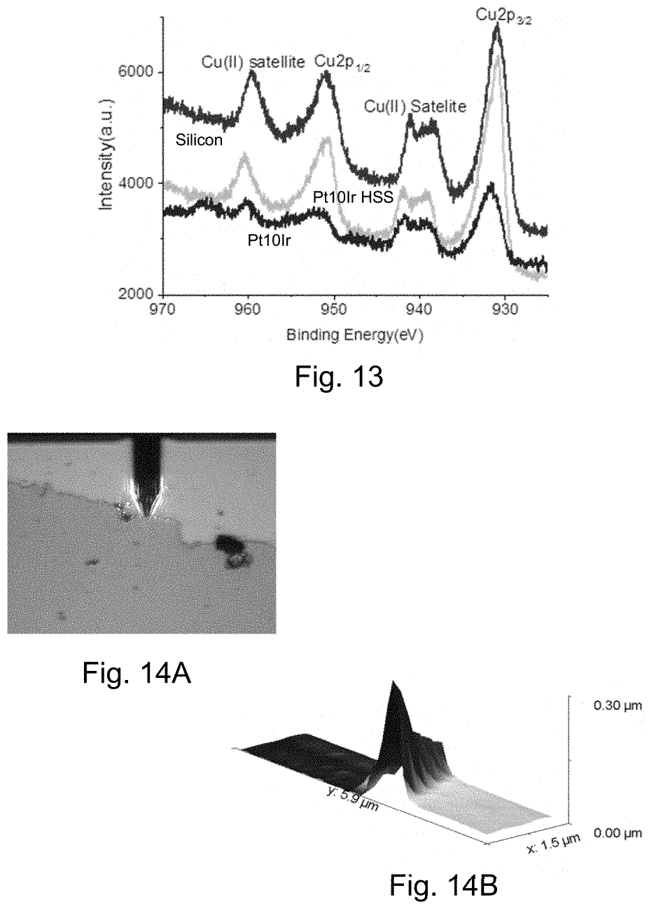

XPS was conducted to check the composition of the O3-ALD copper oxide films. FIG. 13 shows the Cu2p spectra, again indicating oxidation state of Cu2+, i.e., the O3-ALD coated film is CuO.

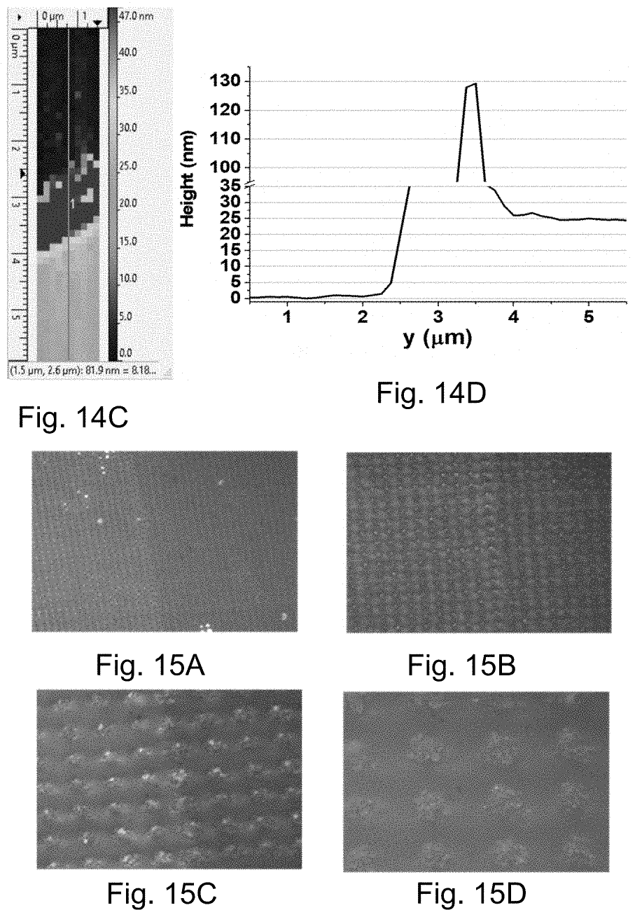

FIGS. 14A-14D shows the AFM scanning of O3-ALD coated Kapton-tape-masked Si sample at the boundary, indicating the film thickness is ˜24 nm, matching well with the ˜22 nm ellipsometry measurement. It is interesting to see the 22 nm coating on Si substrate.

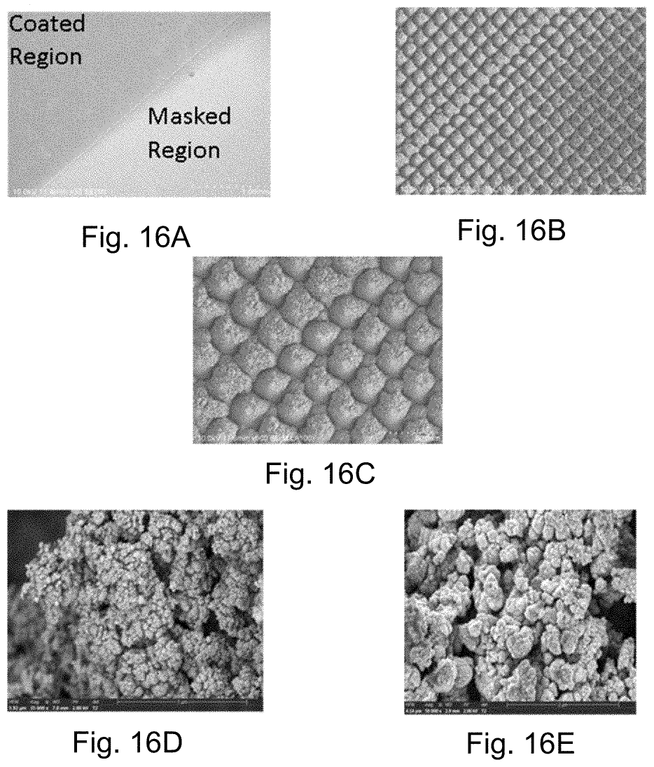

The ˜22 nm O3-ALD CuO coating can be easily identified by optical images, SEM images, and EDS mappings around the masking boundary as in FIGS. 15-17. Optical images (FIGS. 15A-15D) at different magnifications show the color contrast between coated and uncoated region. The coated side looks darker than the uncoated side. SEM images (FIGS. 16A-16E) of the HSS sample show there is sudden change in the structure of the sample around the masked boundary. FIGS. 16D and 16E show that the 22 nm CuO coating nicely and conformally coated the HSS nanostructure, inducing a blunting effect due to the coating compared to the uncoated HSS structure.

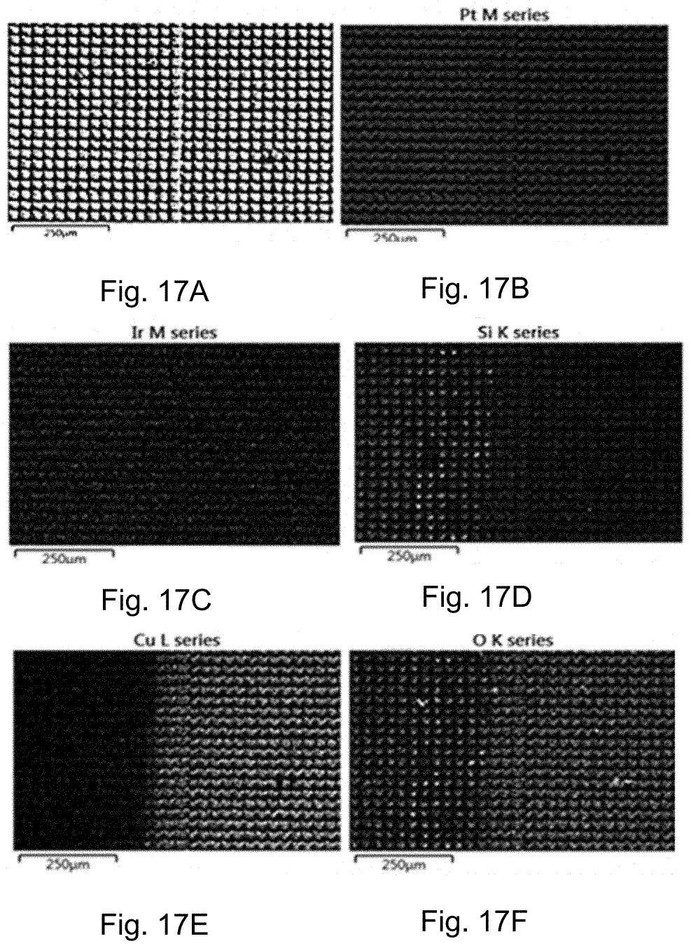

EDS mapping (FIGS. 17A-17F) was performed around the boundary masked and unmasked region where left side is masked, and right side is coated region. It is evident that Platinum and Iridium are present throughout the sample. Cu is found to be present on right side and no Copper is present on the masked region. This is further evidence to prove the efficiency of the masking by Kapton tape on the surface and CuO coating.

Detailed Analysis

Ion release from surfaces—coated and bare substrates (as controls) were tested for static release of copper ions based on the coating composition as provided. The experimental design was as follows: samples were submerged in 10 ml of liquid media (either sterile distilled H₂O or sterile LB media used for bacterial growth) and incubated at 37° C. At time=0 minutes and at 10-minute intervals subsequently, 500 ml aliquots were removed from the solution and immediately mixed with 4.5 ml of 2% nitric acid. These samples were then injected into an ICP-MS tuned for detection of copper ions.

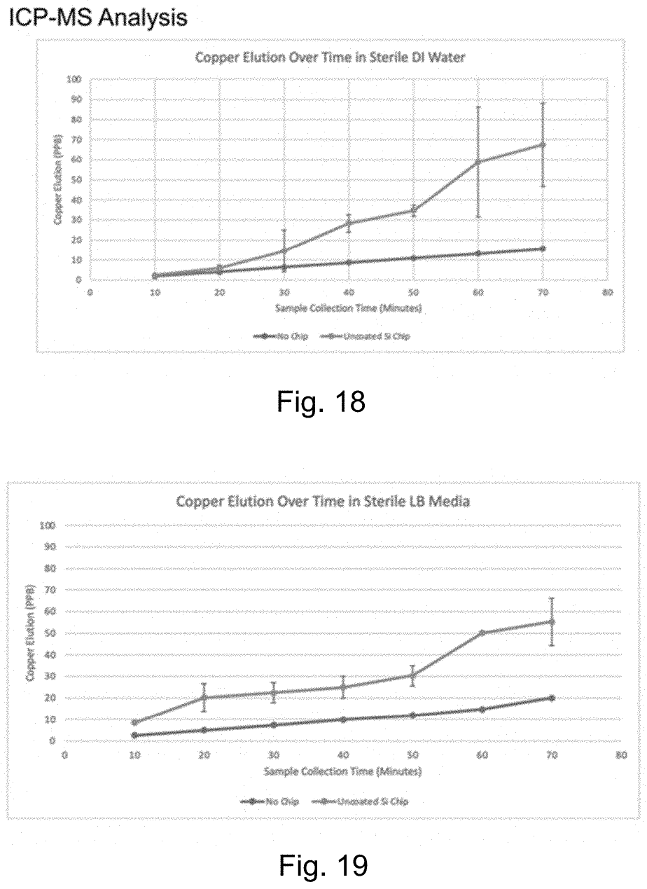

The initial experiment set was performed using silicon substrates without CuO coatings. These results are shown in FIGS. 18 and 19. With the uncoated silicon samples, there was a very low level of Cu found to be present in the samples. These were between 50-70 ppb at the final time point. It is unclear where the Cu ions originated from in these samples, but the signal was consistently 2-3× above background originating from the liquid media.

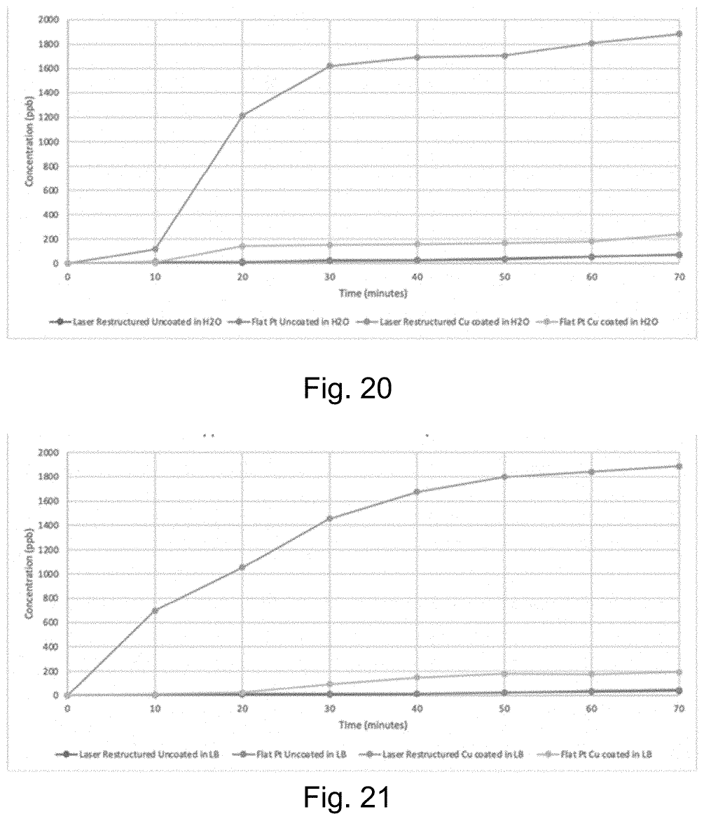

The second experiment set was performed using platinum substrates with and without CuO coatings. This was also diversified by comparing untreated or “flat” Pt substrates with or without CuO coatings to Pt HSS substrates where the surface was laser restructured before coating with CuO. The results of these ICP-MS experiments are shown in FIGS. 20 and 21. These results clearly show the rapid and significant release of Cu ions from the coated, Pt HSS substrates in both water and LB media. This release is an order of magnitude higher when compared to the “flat” Pt substrates (˜1900 ppb vs. ˜200 ppb, at the experimental endpoint). Notably, the CuO coated “flat” substrate did release some Cu ions compared to background (˜200 ppb vs. ˜60 ppb).

Bacterial adhesion—coated materials were tested for bacterial adhesion and contact-dependent antibacterial activity based on the coating composition as provided. The experimental design involved the deposition of a known quantity of bacteria on the surfaces (10 ml of a bacterial culture titered to 1×105 CFU/ml). After incubation for 60 min at 37° C., the surface is swabbed to isolate any remaining viable bacteria using a sterile cotton swab. This swab was then used to inoculate a solid-medium LB-agar plate, and subsequently submerged in 3 ml of sterile LB media for a final inoculation. Both the LB-agar petri dish and the liquid culture were allowed to incubate for 18 h at 37° C., with the liquid samples shaking at 250 rpm to maximize growth. After 18 h, the plates were photographed to determine colony growth while the turbidity of the liquid samples were measured using OD600. The same procedure was used for both bacterial strains of E. coli (Gram negative) and S. aureus (Gram positive).

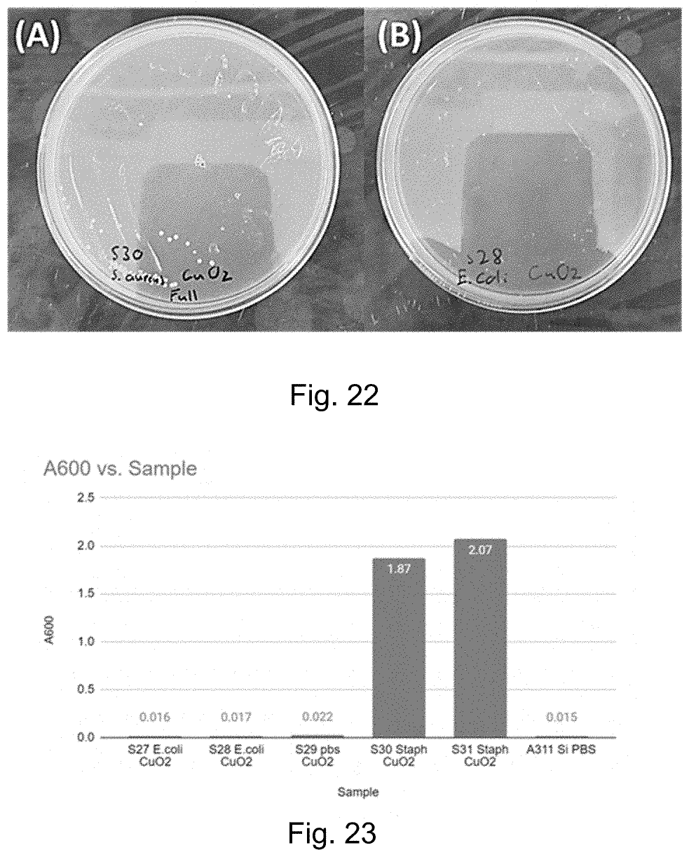

The initial experiment set was performed using silicon substrates with and without CuO coatings. These results are shown in FIGS. 22 and 23. FIG. 22 depicts the agar plates that were inoculated after swabbing CuO coated Si substrates, while FIG. 23 shows the OD600 after inoculation of liquid cultures. The data shows that the E. coli was killed by contact with the CuO surface as evidenced by no colonies appearing on the plate and no observable light scattering in the liquid culture. In contrast, the S. aureus was not eradicated upon exposure to the CuO coated surface as evidenced by the presence of colonies on the petri dish and the high levels of scattering shown in the liquid culture.

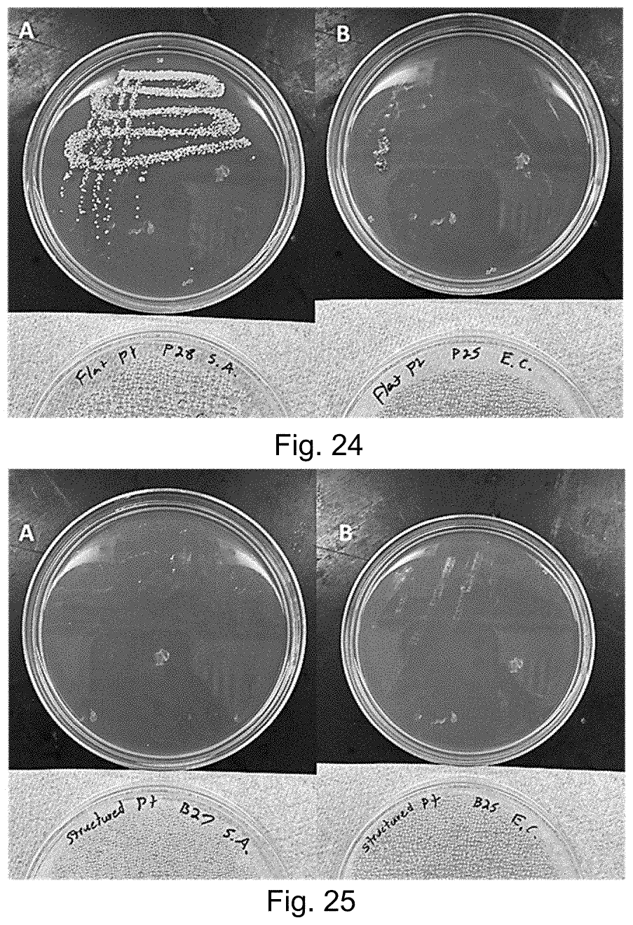

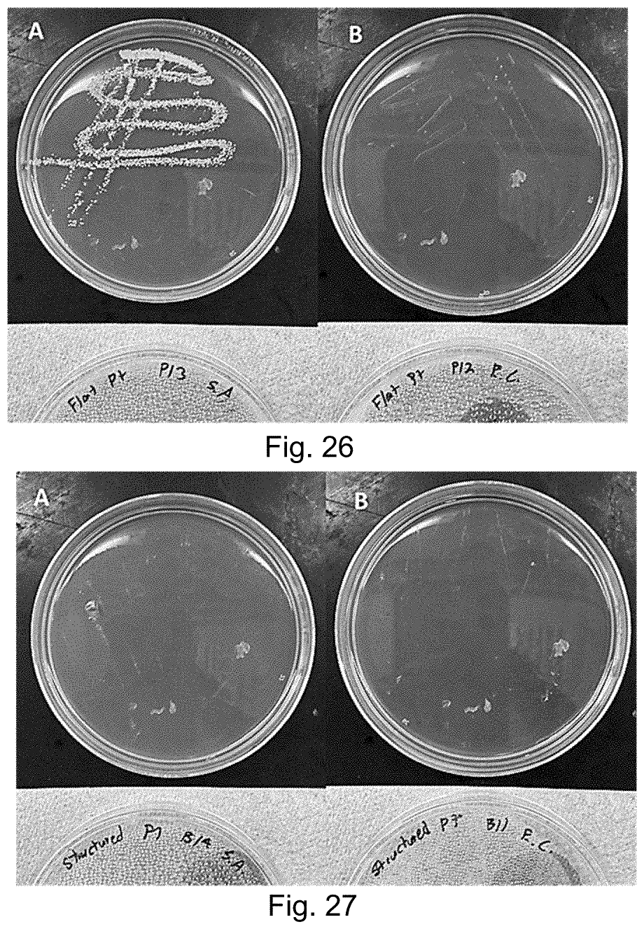

Again, the second set of experiments was performed using platinum substrates with and without CuO coatings. This was also diversified by comparing untreated or “flat” Pt substrates with or without CuO coatings to Pt HSS substrates where the surface was laser restructured before coating with CuO. Results are shown in FIGS. 24-27. In these experiments, E. coli was unable to grow on any of the surface conditions. However, S. aureus was only able to grow on the “flat” surfaces, regardless of the presence or absence of CuO coating. Most importantly, S. aureus showed no colony growth for the samples derived from laser restructured samples, indicating a significant improvement in sterilization by those surfaces.

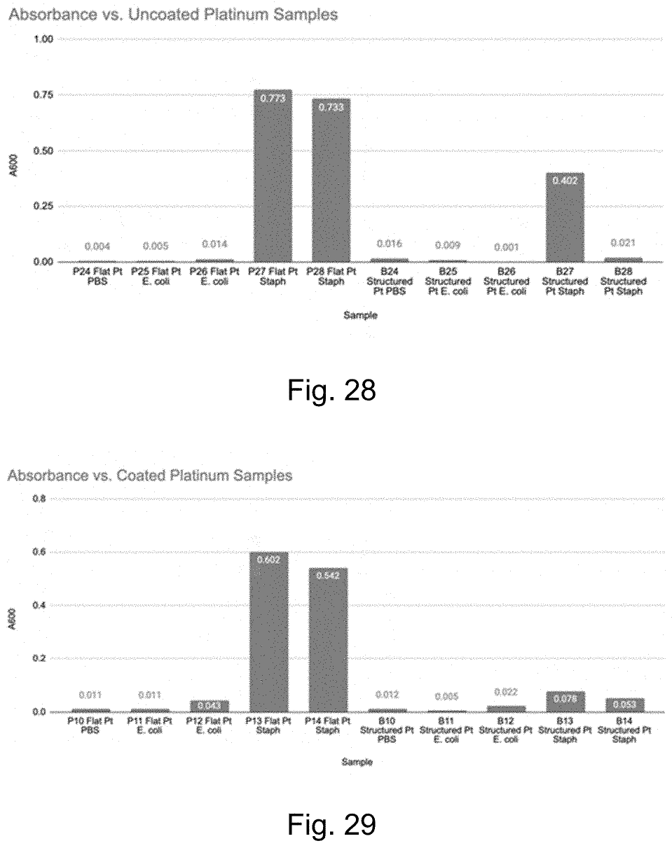

FIGS. 28 & 29 depict the data from the liquid cultures inoculated from the same swabs from FIGS. 24-27. The swabs from the uncoated platinum samples showed the same pattern as found in the agar plate assay with one exception: one culture of S. aureus did show intermediate growth in the liquid culture (FIG. 28). This indicates that there were likely some small number of viable S. aureus cells on the swab (and hence the surface) but those cells were not transferred to the solid medium. However, the coated samples showed identical growth patterns to the plate assay (FIG. 29). Taken together, the data indicate that S. aureus may not be fully eradicated by the uncoated, structured samples, but is fully eradicated by the combination of laser restructuring surfaces AND coating with CuO.

While specific structures and coating patterns are illustrated, the disclosure is not limited to the illustrated embodiments. The laser restructuring or texturing will configure the surface such that the antibacterial/drug eluting coating has the greatest efficacy, for example, having pockets or ledges that protect the coating or delay the exposure/release of the coating. The structure may also be configured to facilitate multiple coatings or mixtures of the oxides or multi-layer coating materials.

The coatings may be applied utilizing various techniques, for example, physical vapor deposition, chemical vapor deposition, or atomic layer deposition. Additionally, the laser restructuring may take place before or after the coating is applied. As one example, a metal coating, e.g. silver or copper, may be applied to the surface using a coating technique and then the laser restructuring is carried out in an oxygen rich environment such that a metal oxide coating is created in-situ during laser restructuring.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed is:

1. A medical device comprising:
a substrate structure with a surface, the surface laser treated to define at least one protrusion and/or at least one void extending relative to the surface; and
a coating having antibacterial, antimicrobial and/or drug eluding properties applied to the substrate structure such that the coating engages within or along a surface portion of one or more of the protrusions and/or voids.

2. The medical device according to claim 1 wherein the surface has a hierarchical laser restructured topography.

3. The medical device according to claim 2 where in the surface topography includes nano protrusions extending from micro protrusions.

4. The medical device according to claim 3 wherein the surface topography is further defined by macro protrusions from which the micro protrusions extend.

5. The medical device according to claim 1 wherein the coating is applied along the entire surface.

6. The medical device according to claim 1 wherein the coating has an atomically thin thickness.

7. The medical device according to claim 1 wherein the coating contains zinc, copper and/or silver.

8. The medical device according to claim 1 wherein the coating contains CuₓO.

9. The medical device according to claim 1 wherein the coating is applied utilizing a deposition process.

10. The medical device according to claim 1 wherein the coating is applied utilizing an atomic layer deposition process (ALD).

11. The medical device according to claim 10 wherein the coating is applied utilizing plasma enhanced ALD or thermal ALD.

12. The medical device according to claim 1 wherein the wherein the substrate structure comprises platinum, steel, an alloy of platinum and iridium, an alloy of nickel and cobalt, titanium, an alloy of titanium, tantalum or combinations thereof.

13. A method of manufacturing a medical device have a substrate structure with a surface, the method comprising:
laser treating the surface to define at least one protrusion and/or at least one void extending relative to the surface; and
applying a coating having antibacterial, antimicrobial and/or drug eluding properties to the substrate structure such that the coating engages within or along a surface portion of one or more of the protrusions and/or voids.

14. The method according to claim 13 wherein the laser treating defines a hierarchical laser restructured topography on the surface.

15. The method according to claim 13 wherein the coating is applied along the entire surface.

16. The method according to claim 13 wherein the coating has an atomically thin thickness.

17. The method according to claim 13 wherein the coating contains zinc, copper and/or silver.

18. The method according to claim 13 wherein the coating is applied utilizing a deposition process.

19. The method according to claim 18 wherein the coating is applied utilizing an atomic layer deposition process (ALD).

20. The method according to claim 19 wherein the coating is applied utilizing plasma enhanced ALD or thermal ALD.
